(19) United States
(12) Patent Application Publication
(10) Pub. No.: US 2025/0352384 A1
(43) Pub. Date: Nov. 20, 2025

(54) A WASTE COLLECTION BAG

(71) Applicant: Salts Healthcare Limited, Birmingham (GB)

(72) Inventor: Ewa Jasicka, Birmingham (GB)

(21) Appl. No.: 18/706,885

(22) PCT Filed: Nov. 4, 2022
(86) PCT No.: PCT/GB2022/052794

§ 371 Date: May 2, 2024

(30) Foreign Application Priority Data
Nov. 5, 2021 (GB) 2115911.6

(51) Int. Cl. A61F 5/44; A61M 1/00

(52) U.S. Cl. CPC A61F 5/4404; A61M 1/69

(57) ABSTRACT

A waste collection bag (10) for connection to an ostomy appliance including: a first wall (12) and a second wall (14), which are connected to form a pouch arrangement including a collecting volume, an inlet (20), for connection to the ostomy appliance, and which inlet permits waste into collecting volume, and a support system (30) having a first attachment zone (32) including an opening (40), a second attachment zone (34) including an opening (42), and a flexible portion (36) wherein the first attachment zone is closer to the collecting volume than the second attachment zone and wherein the flexible portion extends between the first and second attachment zones.

### INTRODUCTION

Embodiments of the invention relate to a waste collection bag.

Ostomy appliances are well known medicals devices for users that have a stoma. Generally, the appliance has an opening for the stoma to be received in and an adhesive member that contacts the body and holds the appliance in position. Waste exiting the stoma is collected in a collecting volume which is formed from two flexible walls connected together. Often the appliance includes an outlet valve, so that a user can empty waste that is collected in the collecting volume without having to remove and dispose of the entire appliance.

At times, a user may want to extend the time between emptying their appliance, for example, when they are sleeping. Typically, a larger capacity bag is used to contain waste while the user is sleeping. In this scenario, the outlet valve is connected to the larger capacity bag by a tube, so that the bag fills with waste.

Embodiments of the present invention seek to alleviate one or more problems associated with the prior art.

According to an aspect of the invention we provide a waste collection bag for connection to an ostomy appliance including:
a first wall and a second wall, which are connected to form a pouch arrangement including a collecting volume,
an inlet, for connection to the ostomy appliance, and which inlet permits waste into collecting volume, and
a support system having:
   a first attachment zone including an opening,
   a second attachment zone including an opening, and
   a flexible portion wherein the first attachment zone is closer to the collecting volume than the second attachment zone and wherein the flexible portion extends between the first and second attachment zones.

According to a second aspect of the invention we also provide a waste collection system including a stand or holder and a waste collection bag in accordance with the first aspect.

Further optional features of the invention are provided in the appended claims.

In order that the present disclosure may be more readily understood, preferable embodiments thereof will now be described, by way of example only, with reference to the accompanying drawings, in which:

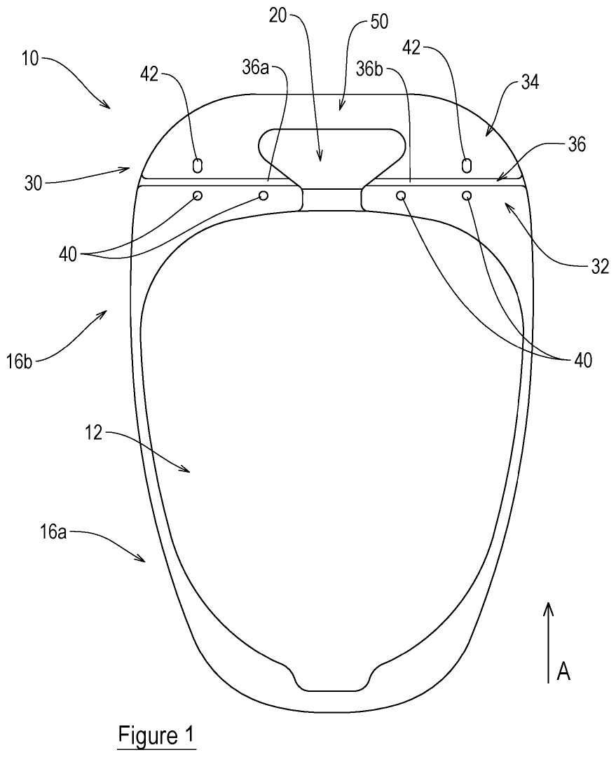

FIG. 1 is a waste collection bag in accordance with embodiment of the present disclosure;

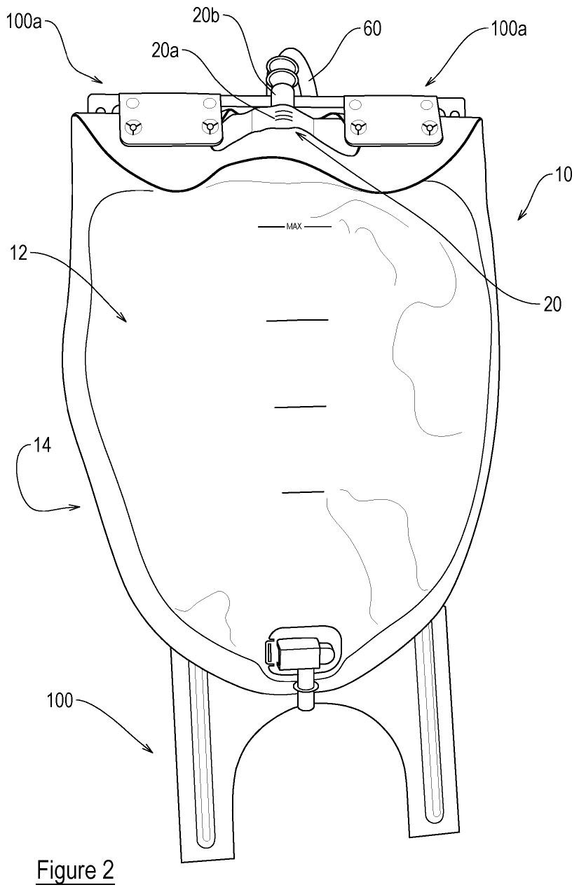

FIG. 2 is the waste collection bag supported on a stand; and

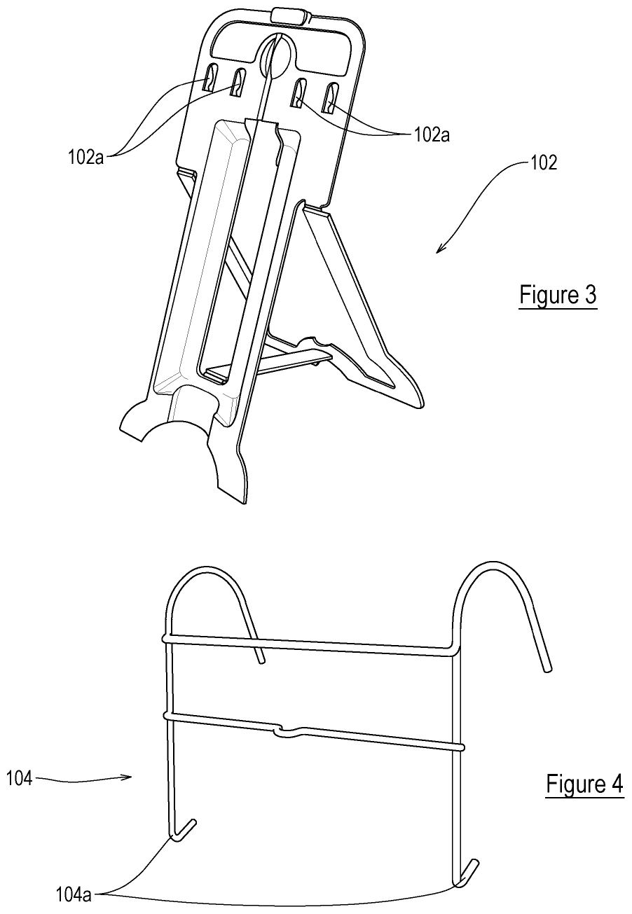

FIGS. 3 and 4 are illustrations of other stand designs that the waste collection is compatible with.

Referring to FIGS. 1 and 2, a waste collection bag 10 for connection to an ostomy appliance (not shown) is illustrated. The waste collection bag 10 includes a first wall 12, a second wall 14, an inlet 20 and a support system 30.

The first wall 12 and the second wall 14 are connected to form a pouch arrangement which includes a collecting volume. In other words, the first and second wall 12, 14 and the connection between them defines and internal volume in which waste is collected. In the present example, the collecting volume is around two litres but it should be appreciated that the bag 10 can be made to form the desired volume within.

In some embodiments, the collecting volume is formed in a lower portion 16a of the pouch arrangement and the support system 30 is formed in an upper portion 16b of the pouch arrangement. In the present example, the collection bag 10 is between about 37 and 38 cm long (and preferably around 37.6 cm long in total and the collecting volume extends from a base of the collection bag 10 upwards, to between about 75% and 85% of the total length. The remaining area at the top of the collection bag 10 (the upper portion) includes the support system 30 that is used to support the collection bag 10 on an appropriate stand, holder or hanger.

The inlet 20 is fluidly connected to the collecting volume, so that waste can flow from a connected device or appliance and into the collecting volume. In use, the inlet 20 is connected to an ostomy appliance (usually a urostomy appliance) via a tube/conduit, so that waste can flow out of a user's appliance (that is adhered about their stoma) and through the connecting tube and into the waste collection bag 10.

In some embodiments, the inlet 20 includes a body having two parts. The first part 20a is attached between the first and second walls 12, 14. The second part 20b extends away from the first part 20a and is configured to connect to a tube/conduit 60.

As can be seen from the illustrated example in FIG. 2, the inlet 20 is provided in a generally central region of the upper portion 16b of the pouch arrangement. In use, the connected tube/conduit 60 extends away from the collecting volume.

The support system 30 includes a first attachment zone 32, a second attachment zone 34, and a flexible portion 36. The first attachment zone 32 includes an opening 40 and the second attachment zone 34 includes an opening 42. Further, the first attachment zone 32 is closer to the collecting volume than the second attachment zone 34. The flexible portion 36 extends between the first and second attachment zones 32, 34. The term flexible portion is used to describe a region that is relatively more flexible than the area or regions around it.

In some embodiments, the first attachment zone 32 includes multiple openings 40. Referring to FIG. 1, the first attachment zone 32 includes four openings 40. The openings 40 are spaced across the upper portion of the pouch arrangement. In the illustrated example, the upper portion 16b of the pouch arrangement is between around 24 and 25 cm wide (and preferably between around 24.6 cm). The “outer” openings 40 at either side of the first attachment zone 32 (i.e. those closest to the edge of the collection bag 10) are around 4 cm from the edge. The “inner” openings 40 are around 4 cm from the outer openings 40. The distance between the “inner” openings 40 is around 8 cm.

In some embodiments, one or more of the openings 40 of the first attachment zone 32 are generally circular (and, in this example, are around 4.5 mm in diameter).

In some embodiments, the second attachment zone 34 includes multiple openings 42. Referring to the example in FIG. 1, the second attachment zone 34 includes two openings 42. The openings 42 are each around 4 cm from the edge of the collection bag 10 and about 16 cm apart. In some embodiments, the openings 42 are slots (i.e. elongate apertures) that are around 6 mm long (i.e. in a lengthwise direction A of the collection bag 10) and 4 mm wide

In some embodiments, the openings 42 of the second attachment zone 34 are substantially aligned with the outer openings 40 of the first attachment zone 32. In other words, in the direction A, the openings 42 of the second attachment zone 34 are above the openings 40 of the first attachment zone 32.

In some embodiments, a handle 50 is provided in the upper portion 16b of the pouch arrangement. In the illustrated example, the handle 50 provides an opening, which forms part of the second attachment zone 34 (the functionality of this is discussed in more detail later).

In some embodiments, the flexible portion 36 extends across the pouch arrangement. In the illustrated example, the flexible portion 36 is split in two parts 36a, 36b. The first part 36a extends from an edge of the upper portion 16b to the opening provided by the handle 50. The second part 36b extends from an opposing side of the opening provided by the handle to a second edge of the upper portion 16b. In other words, the flexible portion 36 extends in a substantially straight line across the upper portion 16b (this is advantageous for the folding functionality provided by the flexible portion 36 as described below). It should be appreciated that in other embodiments, the flexible portion may be formed as a single part extending across substantially the entire width of the upper portion 16b, from one edge to the other.

As the term flexible portion describes a region that is relatively more flexible than the area or regions around it. In some embodiments, the flexible portion 36 is formed by the absence of toughened or thickened material compared to the surrounding area. In this example, the first and second walls 12, 14 are permanently connected together across the area that does not include the collecting volume, which creates a single sheet of material. This creates an upper portion 16b that is reinforced and able to support the weight of the collection bag 10, even when full of waste. However, across the flexible portion 36, the first and second walls 12, 14 are not connected together. This creates a region of comparatively higher flexibility because the two sheets of material are able to bend independently rather than being constrained by having to move together. This is advantageous for allowing the collection bag 10 to adapt to multiple support stands and hangers.

In some embodiments, the first and second walls 12, 14 are welded (preferably heat welded) about their peripheries in the lower portion 16a of the pouch arrangement and the area of the upper portion 16a is more extensively welded in order to provide a support system 30 that is strong/robust enough to support the collection bag 10 (from a hanging position) when full of waste. The flexible portion 36 is a line that is formed across the upper portion 16b between two areas that are substantially completely welded together (i.e. either side of the line is welded across the bag 10). The line remains unwelded (as described as being advantageous above), so that the first and second wall 12, 14 can fold along the line easily. In other words, the flexible portion 36 is provided in a generally straight elongate area across the bag. The relatively less flexible areas are provided both above and below the flexible portion 36. Each of the less flexible areas extends adjacent the flexible portion 36 across the bag 10. It should be appreciated that is the bag 10 includes a handle 50, the flexible portion 36 may be interrupted in a central region for an opening that provides that handle 50. In such an example, the flexible portion 36 and the less flexible areas above and below are formed in two parts that extend either side of the handle 50.

In some embodiments, the flexible portion 36 and the first and second attachment zones 32, 34 are all located in an upper portion of the collection bag 10. In other words, they are all located in a position above the main collecting volume (when the bag 10 is in use). It should be appreciated that the inlet 20 is generally centrally located and may be positioned just below the flexible portion 36.

The waste collection bag 10 is adapted to provide at least two different configurations for connecting to more than one type of support stand, holder or hanger.

FIG. 2 illustrates the collection bag 10 on a “pinch” stand 100. FIGS. 3 and 4 illustrate two other types of stand/hanger that the collection bag 10 is also compatible with.

The stand 100 includes four pins that extend outwards, over which the four openings 40 of the first attachment zone 32 are placed. In other words, the pins extend through the openings 40. The collection bag 10 is folded along the flexible portion 36. This brings the openings 42 of the second attachment zone 34 into contact with the pins also. Thus, the pins extend through the openings 42 of the second attachment zone 34 as well as those of the first attachment zone 32. Further, because the opening provided by the handle 50 flares outwards, this opening is positioned over the pins when the flexible portion 36 is folded. This allows a pinch piece 100a on each side of the stand 100 to be folded down and over the pins to secure the collection bag 10 in place. In other words, the first configuration of the collection bag 10 includes folding along the flexible portion 36, so that both the first attachment zone 32 and the second attachment zone 34 are used to engage the supporting stand 100.

In some embodiments, a stand includes four upward projecting hooks (see the stand 102 illustrated in FIG. 3). There are four hooks 102a along a back plate of the stand 102. In this example, the four openings 40 of the first attachment zone 32 are used to hang the collection bag 10. The remainder of the upper portion 16b of the pouch arrangement is supported by the back plate of the stand 102 and a connected tube 60 is arranged upwards.

Alternative stands with four upward hooks may not include the back plate. In this case, the remainder of the upper portion 16b of the pouch arrangement will bend over the top of the stand.

In some embodiments, a hanger may be used (for example, in a hospital setting where bags are more likely to be hung from the side of a bed). These types of hangers 104 (one of which is shown in FIG. 4), have two wide spaced hooks 104a. The outer openings 40 of the first attachment zone 32 or the openings 42 of the second attachment zone 34 could be used for hanging the collection bag 10 from a two hook hanger. It should be appreciated that if the hanger had four hooks, the opening 40 of the first attachment zone 32 would be compatible with that.

In general terms, the second of the configurations includes using only the first attachment zone 32 or the second attachment zone 34 to engage the supporting stand or hanger 102, 104.

Further, the handle 50 provides an additional way in which the collection bag 10 can be supported. This allows a user to carry the collection bag 10 between locations. Furthermore, the handle 50 provides a larger opening that may allow the user to hang the collection bag 10 when it is not being used. For example, they may wish to hang it in a shower cubicle or another environment when the collection bag 10 has been cleaned and they are allowing it to dry out.

In some embodiments, the material of the waste collection bag 10 preferably does not include polyvinyl chloride (PVC). For example, a suitable material may be polyphenylene ether (PPE) or a thermoplastic polyurethane (TPU) material or a non-coated polyethylene (PE) may be preferred. A suitable PE may be low density polyethylene (LDPE), medium density polyethylene (MDPE), be high density polyethylene (HDPE) or a combination of two or more of them.

In should be appreciated that the first and second wall 12, 14 and the inlet 20 will preferably be made of similar and/or compatible materials. This ensures that the inlet 20 seals to the wall 12, 14 with a reliable and consistent seal when it is welded.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

1. A waste collection bag for connection to an ostomy appliance including:
a first wall and a second wall, which are connected to form a pouch arrangement including a collecting volume,
an inlet, for connection to the ostomy appliance, and which inlet permits waste into the collecting volume, and
a support system having:
a first attachment zone including an opening,
a second attachment zone including an opening, and
a flexible portion wherein the first attachment zone is closer to the collecting volume than the second attachment zone and wherein the flexible portion extends between the first attachment zone and the second attachment zone.

2. The waste collection bag according to claim 1, wherein the first attachment zone and/or the second attachment zone include multiple openings.

3. The waste collection bag according to claim 2, wherein the first attachment zone includes four openings.

4. The waste collection bag according to claim 2, wherein the second attachment zone includes two or three openings.

5. The waste collection bag according to claim 2, wherein the second attachment zone includes multiple openings, and wherein one or more of the openings of the second attachment zone is a slot.

6. The waste collection bag according to claim 1, wherein the collecting volume is formed in a lower portion of the pouch arrangement and the support system is formed in an upper portion of the pouch arrangement.

7. The waste collection bag according to claim 6, wherein the upper portion includes a handle.

8. The waste collection bag according to claim 7, wherein the handle includes an opening in the upper portion.

9. The waste collection bag according to claim 8, wherein the handle opening forms one or more of the openings of the second attachment zone.

10. The waste collection bag according to claim 1, wherein the flexible portion extends across the pouch arrangement.

11. The waste collection bag according to claim 8, wherein the flexible portion is split into a first part and a second part, and the first part extends from a first edge of the upper portion to the handle opening and the second part extends from an opposing side of the handle opening to the first edge of the upper portion or to a second edge of the upper portion.

12. The waste collection bag according to on claim 6, wherein the upper portion is formed from the first wall and the second wall being permanently attached together and the flexible portion is a region in which the first wall and the second wall are not connected.

13. The waste collection bag according to claim 12, wherein the permanent attachment is formed by a heat welding process.

14. The waste collection bag according to claim 1, wherein the inlet includes a body, which includes a first part attached between the first wall and the second wall and a second part that extends away from the first part and is configured to connect to a conduit.

15. The waste collection bag according to claim 14, wherein the inlet is provided in a generally central region of an upper portion of the pouch arrangement, so that when the conduit is attached, the conduit extends away from the collecting volume.

16. The waste collection bag according to claim 1, including at least two different configurations for connecting to more than one type of supporting stand, holder, or hanger.

17. The waste collection bag according to claim 16, wherein one of the at least two different configurations includes a fold along the flexible portion, so that both the first attachment zone and the second attachment zone are used to engage the supporting stand, holder, or hanger.

18. The waste collection bag according to claim 16 wherein one of the at least two different configurations includes using only the first attachment zone or the second attachment zone to engage the supporting stand, holder, or hanger.

19. The waste collection bag according to claim 16, wherein an additional hanging configuration is provided by a handle.

20. A waste collection system including a stand or holder and a waste collection bag according to claim 1.
